(19) United States
(12) Patent Application Publication
(10) Pub. No.: US 2023/0149504 A1
(43) Pub. Date: May 18, 2023

(54) ALISPORIVIR FOR USE IN HUMAN VIRAL INFECTIONS

(71) Applicants: DEBIOPHARM INTERNATIONAL SA, Lausanne (CH); INSERM (INSTITUT NATIONAL DE LA SANTÉ ET DE LA RECHERCHE MÉDICALE), Paris (FR); UNIVERSITÉ PARIS EST CRÉTEIL VAL DE MARNE, Créteil (FR); ASSISTANCE PUBLIQUE - HÔPITAUX DE PARIS, Paris (FR)

(72) Inventors: Jean-Michel PAWLOTSKY, Sèvres (FR); Abdelhakim AHMED-BELKACEM, Ivry-sur-Seine (FR); Laurent SOFTIC, Issy-les-Moulineaux (FR); Valérie NICOLAS, St-Prex (CH); George BADITA, Stansstad (CH); Raf CRABBE, Bassins (CH)

(21) Appl. No.: 17/995,527

(22) PCT Filed: Apr. 6, 2021
(86) PCT No.: PCT/IB2021/052846

§ 371 Date: Oct. 5, 2022

(30) Foreign Application Priority Data
Apr. 6, 2020 (WO) PCT/IB2020/053266

(51) Int. Cl. A61K 38/13; A61P 31/14

(52) U.S. Cl. CPC A61K 38/13; A61P 31/14

(57) ABSTRACT

The present invention concerns alisporivir for use in treatment of a human patient suffering of COVID-19 infections and for prevention of a human patient from suffering of COVID-19 infections.

The present invention concerns the drug alisporivir for use in the treatment of a human patient suffering of COVID-19 infections and for prevention of a human patient from suffering of COVID-19 infections.

Coronaviruses are a large family of viruses that usually cause medium to moderate upper-respiratory tract illnesses, like the common cold, in people. However, three times in the 21st century coronavirus outbreaks have emerged from animal reservoirs to cause severe disease and global transmission concerns. There are hundreds of coronaviruses, most of which circulate among animals including pigs, camels, bats and cats. Sometimes those viruses jump to humans (process called a spillover event) and can cause disease. Seven coronaviruses are known to cause human disease, four of which are medium: viruses 229E, 0043, NL63 and HKU1.

Three of the coronaviruses can have more serious outcomes in people, and those diseases are SARS (severe acute respiratory syndrome), caused by coronavirus known as SARS-Cov-1, which emerged in late 2002 and disappeared by 2004; MERS (Middle East respiratory syndrome), which emerged in 2012 and remains in circulation in camels; and the new coronavirus disease 2019 (COVID-19), which emerged in December 2019 from China and a global effort is under way to contain its spread. COVID-19 is caused by the coronavirus known as SARS-CoV-2.

The WHO reports that human-to-human transmission is occurring with a preliminary RO estimate of 1.4-2.5. Current estimates of the incubation period of the virus range from 2-14 days, and the virus seems to be transmitted mainly via respiratory droplets when people sneeze, cough, or exhale.

Typical symptoms of COVID-19 include fever, cough, difficulty breathing, muscle pain and tiredness. More serious cases develop severe pneumonia, acute respiratory distress syndrome, sepsis and septic shock through bacterial superinfections. Generally, older people and those with underlying conditions (such as hypertension, heart disorders, diabetes, liver disorders, and respiratory diseases) are expected to be more at risk of developing severe symptoms. The evidence from analyses of cases to date is that COVID-19 infection causes medium disease (i.e. non-pneumonia or medium pneumonia) in about 80% of cases and most cases recover; 14% have more severe disease and 6% experience critical illness.

Phylogenetic analyses of 15 HCoV whole genomes reveal that 2019-nCoV/SARS-CoV-2 shares the highest nucleotide sequence identity with SARS-CoV-1 (79.7%). Specifically, the envelope and nucleocapsid proteins of 2019-nCoV/SARS-CoV-2 are two evolutionarily conserved regions, having the sequence identities of 96% and 89.6%, respectively, compared to SARS-CoV.

Alisporivir (INN) ([D-MeAla]3-[EtVal]4-CsA; CAS RN 254435-95-5) is cyclic undecapeptide which is synthesized from cyclosporine A (WO 00/01715). It differs from parent cyclosporine A, with sarcosine replaced by Me-alanine at position 3, with leucine replaced by valine at position 4, and with the nitrogen being N-ethylated instead of N-methylated. These modifications enhance the binding affinity of alisporivir for cyclophilins while abolishing its binding to calcineurin and thus immunosuppressive activity.

Alisporivir clears HCV replicon cells when used alone or in combination with direct-acting antivirals, such as protease or polymerase inhibitors (Paeshuyse J et al, Hepatology 2006: 43, 761-770; Gallay et al., Drug Design, Development and Therapy 2013:7 105-115) and has been tested in more than 2000 patients suffering of chronic hepatitis C (Stanciu C et al, Exp Op Pharmacother, 2019:20 379-384). Cyclophilin A is the principal cyclophilin that is essential for HCV viral replication, and its blockade underlines the anti-HCV activity of cyclophilin inhibitors.

Alisporivir shows preclinical activities against some coronaviruses such as Severe Acute Respiratory Syndrome-Corona Virus (SARS-CoV-1), Middle East Respiratory Syndrome virus (MERS-CoV), feline Coronavirus (FCoV), or Mouse Hepatitis Virus (MHV-LUC) (WO 2015/161908), or HCoV-229E (Ma-Lauer et al., Antiviral Research 173 (2020) 104620). So far, the EC50 of alisporivir in vitro against different SARS-Cov-1 ranged between 2 and 10 microM, 10.-50 fold higher than for HCV replicon (de Wilde A H et al, Virus Research, 2017:228, 7-13). The strong involvement of cyclophilin A in the replication of CoV-1 is still controversial and its possible role remains unknown for SARS-CoV-2.

Considering the rapid and global spread of SARS-CoV-2 and the severity of COVID-19 infections and theft high mortality, there is an urgent need to provide the clinician with a medication for use in the treatment of human patient suffering of COVID-19 infections.

The aim of the present invention is to provide such a medication that reduces the SARS-CoV-2 viral load, improves the clinical symptoms, reduces the need for Intensive Care Unit admission and procedures and ultimately help curing the infection and reduce the mortality associated with the COVID-19 infection.

It has been surprisingly found that alisporivir can be used for the treatment of a human patient suffering of COVID-19 infections.

The present invention concerns the drug alisporivir for use in treatment of a human patient suffering of COVID-19 infections, initially known as Coronavirus disease 2019 and for prevention of a human patient from suffering of COVID-19 infections. These infections are due to SARS-CoV-2, formerly called 2019-nCoV.

The present invention further concerns the drug alisporivir for use in prevention of a human patient from suffering of COVID-19 infections once a positive test for SARS-CoV has been performed.

The present invention concerns also alisporivir for use in the treatment of human patients suffering of COVID-19 infections for reducing SARS-CoV-2 viral load. Said SARS-CoV-2 is located in the airways, mainly in the lower airways, in particular in lung.

According to the present invention, alisporivir is administered at a total dose comprised between 200 mg and 1500 mg per day. For example, alisporivir is administered at a total dose of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg or 1500 mg per day. Preferably alisporivir is administered at a total dose comprised between 400 mg and 1200 mg per day. More preferably alisporivir is administered at a daily dose comprised between 800 mg and 1200 mg per day. For example, alisporivir is administered at a daily dose of 800 mg, 850 mg, 900 mg, 950 mg, 1000 mg, 1050 mg, 1100 mg, 1150 mg or 1200 mg.

Alisporivir is administered once a day (Quaque Die, QD), or twice a day (Bis In Die, BID), or three times a day (Ter In Die, TID), or four times a day (Quater In Die, QID).

Preferably, alisporivir is administered at a daily regimen of 800 mg to 1200 mg as total dose per day, administered one or two times a day (i.e. 400 mg to 600 mg Bis In Die (BID)).

Alisporivir is administered through oral route or naso-gastric intubation route for patients not able to swallow voluntarily due to their medical condition.

When alisporivir is administered through oral route, the following pharmaceutical formulations may be used:
a) Oral solution

| | | | Quantity | |
|---|---|---|---|---|
| | Reference to | | per 100 g | Theoretical |
| | Quality | | bottle | Percentage |
| Component | Standard | Function | (g) | (% w/w) |
| Alisporivir | internal | Active | 10.0 | 10.0 |
| | | substance | | |
| Macrogolglycerol | Ph. Eur/ | Solubilizer/ | 41.0 | 41.0 |
| Hydroxystearate | NF | Emulsifier | | |
| Glycerol | Ph. Eur/ | Solubilizer | 32.0 | 32.0 |
| Monolinoleate | NF | | | |
| Ethanol, | Ph. Eur/ | Solvent | 9.0 | 9.0 |
| anhydrous | USP | | | |
| Propylene Glycol | Ph. Eur/ | Solvent | 8.0 | 8.0 |
| | USP | | | |
| TOTAL | | | 100.0 | 100.0 |

b) Capsules

| | Batches | | | | |
|---|---|---|---|---|---|
| | No 1 | | | No 2 | |
| | % | mg/capsule | | % | mg/capsule |
| Fill | | | | | |
| Alisporivir | 13.7% | 200.0 | mg | 13.5% | 200.0 mg |
| Ethanol | 17.8% | 260.0 | mg | 17.5% | 260.0 mg |
| Labrasol | 17.2% | 250.0 | mg | 16.9% | 250.0 mg |
| TPGS | 10.3% | 150.0 | mg | 10.1% | 150.0 mg |
| Labrafac | 3.4% | 50.0 | mg | 3.4% | 50.0 mg |
| Montane 80 | 10.3% | 150.0 | mg | 10.1% | 150.0 mg |
| Shell | | | | | |
| Gelatin** | 20.8% | 302.9 | mg | 20.0% | 296.6 mg |
| Glycerin** | 6.1% | 88.4 | mg | 8.1% | 119.9 mg |
| TiO2 | 0.4% | 6.3 | mg | 0.4% | 6.3 mg |

According to the present invention, alisporivir is administered daily for up to 21 days, preferably up to 14 days.

According to the present invention, the patients are patients suffering from medium to severe COVID-19 infections. Definitions of different degrees of severity of illness can be found in WHO Interim guidance, Clinical management of severe acute respiratory infection (SARI) when COVID-19 disease is suspected, dated Mar. 13, 2020.

In particular, to be eligible for the treatment, patients with medium/severe COVID-19 infections should meet the following criteria:
a) have a diagnosis of COVID-19 infection with an onset of symptoms and a positive PCR test for SARS-CoV-2 within 48 hours prior hospitalisation;

and at least one of the following criteria:
b) radiographic infiltrates by imaging (chest x-ray, CT scan, etc.);
c) clinical assessment (evidence of rales/crackles on exam) and SpO2<94% on room air; or
d) requiring supplemental oxygen.

As evident, prevention of a human patient from suffering of COVID-19 infections includes reducing the risk of the patient suffering from medium to severe COVID-19 infections.

The present invention will be explained in more detail with the following figures and examples.

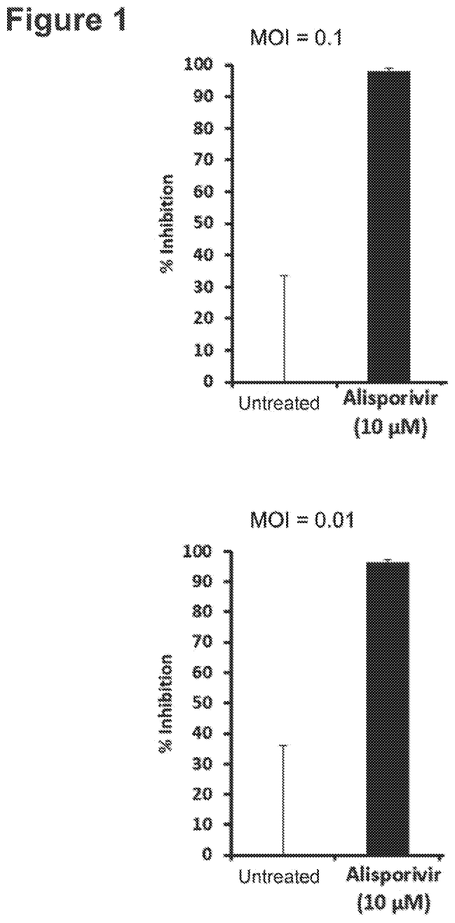

FIG. 1 represents the percentage of inhibition of SARS-CoV-2 RNA production in cell culture supernatant of alisporivir at 10 microM at two Multiplicity of Infections (MOI), respectively 0.1 and 0.01.

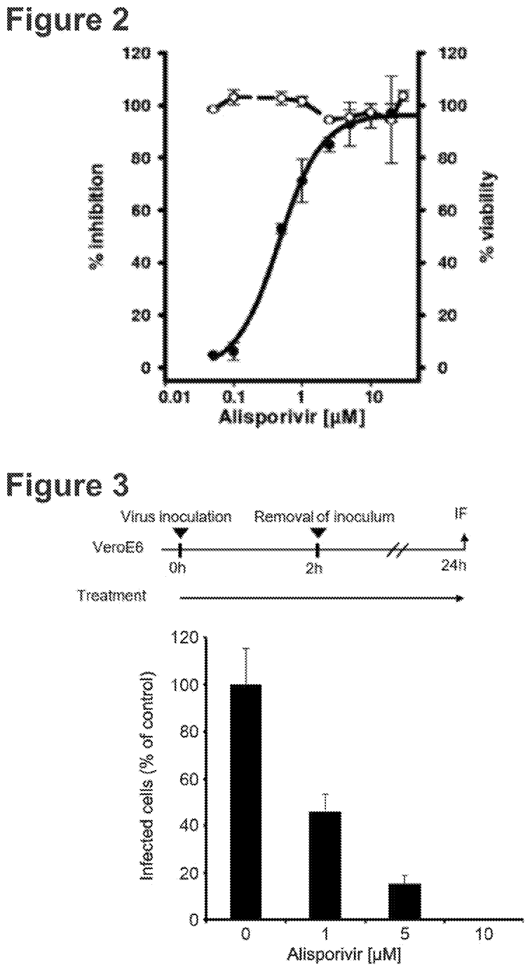

FIG. 2 represents the percentage of inhibition of SARS-CoV-2 RNA production in cell culture supernatant of increasing concentrations of alisporivir at an MOI of 0.02 (solid line) and the cell viability (dashed line).

FIG. 3 represents the experiment scheme of a SARS-CoV-2 infection of Vero E6 cells at an MOI of 0.4 and the percentage of infected cells assessed by immunofluorescence (IF) in the presence of increasing concentrations of alisporivir.

### EXAMPLE 1A: PRECLINICAL ASSAY—INHIBITION OF SARS-COV-2 RNA PRODUCTION

VeroE6 cells were infected with SARS-Cov-2 (isolate from patient infected in Paris), alisporivir diluted in culture medium was added at different concentrations and the cells were incubated for five days in a humidified incubator at 37° C. and 5% Co2.

The antiviral effect was assessed by measuring the SARS-Cov-2 RNA relative quantities in the cell supernatant. For cytotoxicity, cells were incubated with serial alisporivir dilutions in the absence of virus challenge. Data analysis and statistics were performed with Prims and SigmaPlot software.

The Half Maximal Effective Concentration (EC50) of alisporivir on the percentage of SARS-Cov-2 RNA production in cell culture supernatant was 0.54+/−0.06 microM. The compound was not cytotoxic at 20 microM. The index of selectivity (CC50/EC50) of alisporivir was >37.

In FIG. 1, the percentage of inhibition of SARS-CoV-2 RNA production in cell culture supernatant of alisporivir at 10 microM at two MOI (0.1 and 0.01) was reported and was >90%.

### EXAMPLE 1B: DOSE-DEPENDENT ANTIVIRAL ACTIVITY OF ALISPORIVIR AGAINST SARS-COV-2 IN VITRO

The goal of this study was to assess the antiviral properties of alisporivir against SARS-CoV-2, with the objective of generating the preclinical proof of concept of antiviral effectiveness required to start a clinical trial in patients with COVID-19.

The antiviral effectiveness of increasing concentrations of alisporivir was measured in Vero E6 cells infected for 2 h with a clinical isolate of SARS-CoV-2 at a multiplicity of infection (MOI) of 0.02 (FIG. 2). Dimethyl sulfoxide (DMSO) was used as a negative control of antiviral inhibition. Alisporivir was added at the beginning of infection, and viral RNA was extracted from supernatant at 48 h post infection and quantified by reverse transcriptase quantitative PCR (RT-qPCR).

Alisporivir reduced SARS-CoV-2 RNA production in a dose-dependent manner: the 50% effective concentration (EC50) was 0.46+/−0.04 μM, and the EC90 was 3.10+/−1.40 μM. The maximum viral RNA reduction was 2 log 10 at 5 μM. Alisporivir was not cytotoxic at the effective concentration, with a 50% cytotoxic concentration (CC50s) of more than 20 μM and a therapeutic index of more than 43.

The anti-SARS-CoV-2 effectiveness of alisporivir was confirmed by immunofluorescence. Vero E6 cells were infected by SARS-CoV-2 at an MOI of 0.4 for 2 h in the presence of increasing concentrations of alisporivir (1, 5 and 10 μM). After virus removal, infected cells were incubated for 24 h in the presence of alisporivir and immunostained with an anti-double-stranded-RNA (dsRNA) antibody. Infected cells were quantified using ImageJ software. Alisporivir reduced the number of SARS-CoV-2-infected cells in a dose-dependent manner, and complete inhibition was attained at 10 μM (FIG. 3).

### EXAMPLE 2: RANDOMIZED, OPEN-LABEL, PHASE 2 STUDY

The aim of this study is to evaluate the efficacy, safety, tolerability, of alisporivir in combination of Standard of Care (SOC) as compare to SOC alone for the treatment of hospitalized patients with medium to severe infections due to SARS-CoV-2 (COVID-19 infections), excluding patients with Acute Respiratory Distress Syndrome and/or need for mechanical ventilation. The study objectives are a) to evaluate the reduction in SARS-CoV-2 viral load in naso-pharyngeal swabs at Day7 in patients treated with alisporivir in combination with SOC compared to patients treated with SOC alone in patients hospitalized for medium to severe COVID-19 infections, b) to compare the percentage of patients on mechanical ventilation at Day 14.

The study population is hospitalized adults with a diagnosis of COVID-19 infection in its early stage (symptoms onset and COVID-19 RT-PCR test positive in naso-pharyngeal swab within the last 48 hours. A total of 81 subjects are enrolled and randomized 2:1 in one of the two treatment arms (54 subjects in the alisporivir combination with SOC treatment arm and 27 subjects in the SOC treatment arm).

Alisporivir is administered at a dose of 600 mg p.o. BID from Day 1 to Day 14 with the possibility to make dose adjustments, the dosing regimen for alisporivir of 600 mg twice daily for 14 days being selected based on available PK and safety results in healthy volunteers and patients infected with Hepatitis C Virus (HCV), lung penetration data obtained in rat studies and in vitro activity of alisporivir against Sars-CoV-2.

During a weekly Safety Monitoring Committee meeting, decision is taken on early stopping for futility or safety. If a significant beneficial effect of alisporivir is observed, patients on SOC alone could be switched to the alisporivir+SOC arm.

Eligible patients have the onset of symptoms AND a laboratory-confirmed SARS-CoV-2 infection as determined by polymerase chain reaction (PCR) in a naso-pharyngeal swab collected 48 hours prior to randomization. All patients have the RT-PCR COVID19 test based on the same validated method for the whole duration of study in order to ensure consistency and reliability. Sample collection and processing for determining the viral load are done in a standardized and consistent manner in order to decrease variability between samples that might influence the viral load. It is envisaged to use a quantitative method assessing the average viral load/infected host cell, accounting the number of cells from each swabbing sample and the viral RNA copies/cell (“Droplet Digital PCR”).

While it has been shown that alisporivir can be administered safely up to 48 weeks according to previous studies in Chronic Hepatitis C, alisporivir is administered up to 14 days with the possibility to increase the duration of administration to 21 days or more, as required by the clinical and safety parameters.

Participants will be randomized 2:1 into one of the following treatment arms:
Treatment Arm 1: Alisporivir+SOC (N=54): Fifty four (54) participants are enrolled and randomized to receive Alisporivir 600 mg p.o. BID plus SOC from Day1 to Day 14. Total duration of treatment: 14 days.
Treatment Arm 2: SOC (N=27): Twenty seven (27) participants are enrolled and randomized to receive SOC for 14 days.

For the SOC, the investigator chooses the treatment based on locally accepted regimen protocols for patients care and select agents based on the underlying diagnosis and the severity of Covid-19 infection. Additions or changes to SOC are allowed during the patient participation in the study based on patient status and evolution, including antibiotics (excepting Azithromycin due to drug-drug interaction with Alisporivir). Prohibited medications are all anti-viral agents, immunomodulators including Interferons and corticosteroids, Azithromycin, mABs (e.g. tocilizumab), ACE inhibitors, immunoglobulins as well as any experimental drugs.

De-escalation (discontinuing a SOC agent if no longer needed—e.g. administration of oxygen) is allowed and is not considered a failure. Treatment beyond 14 days is considered a treatment failure.

SARS-CoV-2 viral load assessments and Clinical Assessments will occur daily from Day 1 to Day 14 and then until discharge from hospital.

Participants are screened within 48 hours prior to dosing. All participants are treated with alisporivir+SOC or SOC alone up to 14 days. The maximum duration of participation for each participant is approximately 32 days (2-day screening period, 14-day treatment period, and 16-day follow-up period). The patients remain hospitalized for the whole duration of treatment and discharged from hospital based on the investigator's assessment of their status.

The inclusion criteria are, in particular, the followings:
1) hospitalized at the time of enrolment.
2) have a diagnosis of Covid-19 infection with an onset of symptoms and a positive PCR test within the prior 48 hours.
3) have at least one (1) at least one of the following:
   a) radiographic infiltrates by imaging (chest x-ray, CT scan, etc.); or
   b) clinical assessment (evidence of rales/crackles on exam) and SpO2</=94% on room air; or
   c) requiring supplemental oxygen.

The exclusion criteria are, in particular, the followings:
1) concomitant severe bacterial infection including blood stream infections, endocarditis, osteomyelitis, retroperitoneal abscess, septic arthritis, or meningitis diagnosed within 7 days prior to randomization.
2) any of the following signs of severe sepsis:
   a) shock or profound hypotension defined as systolic blood pressure <90 mm Hg or a decrease of >40 mm Hg from baseline that is not responsive to fluid challenge,
   b) hypothermia (core temperature <35.6° C. or <96.1° F.),
   c) disseminated intravascular coagulation (DIC) as evidenced by PT, PTT 2× upper limit of normal, or platelets <50% of the lower limit of normal.
3) history of positive test for human immunodeficiency virus (HIV) including all patients currently on HAART regardless the CD4 count.
4) presence of immunodeficiency or an immunocompromised condition including neutropenia (<1,000 neutrophils/mm³ obtained from the local laboratory at Screening), hematologic malignancy, history of hematopoietic stem cell transplant, history of solid organ transplant, receiving immunosuppressive therapy (e.g. cancer chemotherapy, monoclonal antibodies for autoimmune disease, or medications to prevent transplant rejection), and long-term use of systemic corticosteroids (e.g., ≥20 mg/day of prednisone or systemic equivalent for at least 2 weeks);
5) severe hepatic impairment at Screening, as evidenced by alanine aminotransferase (ALT) or aspartate aminotransferase (AST)>5× upper limit of normal (ULN) or total bilirubin >3×ULN, or clinical signs of cirrhosis or end-stage hepatic disease (e.g., ascites, hepatic encephalopathy);
6) patient has acute hepatitis, cirrhosis (any Child-Pugh class), acute hepatic failure, or acute de-compensation of chronic hepatic failure;
7) alkaline phosphatase >3.0×ULN. Patients with values >3.0×ULN and <5.0×ULN are eligible if this value is documented to be acute and directly related to the infectious process being treated.
8) severe renal impairment (Creatinine-clearance <30 ml/min) or end-stage renal disease (ESRD) requiring haemodialysis or peritoneal dialysis.
9) history of known or suspected Clostridium difficile infection.
10) history of epilepsy or known seizure disorder (excluding a history of childhood febrile seizures).
11) acute co-morbidity within 7 days before inclusion such as myocardial infarction
12) patients previously treated with antivirals, immunomodulators and other medicines prohibited in this study (i.e. remdesivir, interferon ß-1a, lopinavir/ritonavir, hydroxychloroquine etc) in the 14 days prior enrolment.

Assessment of clinical status change based on Percentage of subjects reporting each severity rating on an 8-point ordinal scale [Time Frame: daily from Day1 to Hospital Discharge and then at FUP visits]

The ordinal scale is an assessment of the clinical status at the first assessment of a given study day. The scale is as follows:
1) death.
2) hospitalized, on invasive mechanical ventilation or extracorporeal membrane oxygenation (ECMO);
3) hospitalized, on non-invasive ventilation or high flow oxygen devices.
4) hospitalized, requiring supplemental oxygen.
5) hospitalized, not requiring supplemental oxygen—requiring ongoing medical care (COVID-19 related or otherwise);
6) hospitalized, not requiring supplemental oxygen—no longer requires ongoing medical care.
7) not hospitalized, limitation on activities and/or requiring home oxygen.
8) not hospitalized, no limitations on activities.

To be eligible for study enrolment and randomization, a subject must have at least one prior test positive for SARS-CoV-2 within 48 h. Viral load in nasopharyngeal swabs is performed daily until discharge from the hospital/withdrawal from the study and at the FUP visits. All patients have the RT-PCR COVID19 test based on the same validated method for the whole duration of study in order to ensure consistency and reliability. Sample collection and processing for determining the viral load are done in a standardized and consistent manner in order to decrease variability between samples that might influence the viral load. It is envisaged to use a quantitative method assessing the average viral load/infected host cell, accounting the number of cells from each swabbing sample and the viral RNA copies/cell.

The primary efficacy endpoints are the proportion of patients achieving Viral Load Response Rate (VLRR) response at Day 7, Viral Load Response being defined as an intra patient decrease of 1.5 in log₁₀ viral load compared to baseline.

The secondary efficacy endpoints are:
1) all cause mortality at EOT, TOC, and FU;
2) clinical Efficacy: Percentage of subjects reporting each severity rating on an 8-point ordinal scale [Time Frame: daily from Day1 to Hospital Discharge and then at FUP visits]— see above the 1-8 scale.
3) change in National Early Warning Score (NEWS) from baseline [Time Frame: Day 1 through Day 29]. The NEW score has demonstrated an ability to discriminate patients at risk of poor outcomes. This score is based on 7 clinical parameters (respiration rate, oxygen saturation, any supplemental oxygen, temperature, systolic blood pressure, heart rate, level of consciousness). The NEW Score is being used as an efficacy measure.
4) percentage of patients admitted to ICU and Time to admission to ICU
5) percentage of patients requiring mechanical ventilation
6) time to negative viral load
7) time to resolution of symptoms
8) duration of need for supplemental oxygen
9) safety:
   a) cumulative incidence of Grade 3 and 4 adverse events (AEs) [Time Frame: Day 1 through Day 28±2 days]
   b) cumulative incidence of serious adverse events (SAEs) [Time Frame: Day 1 through Day 28±2 days]
10) duration of hospitalization [Time Frame: Day 1 through Day 28±2 days]-Measured in days.
11) duration of new non-invasive ventilation or high flow oxygen use [Time Frame: Day 1 through Day 28±2 days]
12) duration of new oxygen use [Time Frame: Day 1 through Day 28±2 days]
13) duration of new ventilator or extracorporeal membrane oxygenation (ECMO) use [Time Frame: Day 1 through Day 28±2 days]
14) number of non-invasive ventilation/high flow oxygen free days [Time Frame: Day 1 to Day 28±2 days]
15) number of oxygenation free days [Time Frame: Day 1 to Day 28±2 days]
16) subject 14-day mortality [Time Frame: Day 1 through Day 14]: Date and cause of death (if applicable).
17) subject 28-day mortality [Time Frame: Day 1 through Day 28±2 days] Date and cause of death (if applicable).

### EXAMPLE 3: RANDOMIZED, OPEN-LABEL, PROOF OF CONCEPT, PHASE 2 STUDY

This is a randomised, open-label, proof of-concept, Phase 2 study to evaluate the efficacy, safety and tolerability of alisporivir plus Standard of Care (SOC) as compared to SOC for the treatment of hospitalised patients with infections due to SARS-CoV-2 (COVID-19), excluding patients with acute respiratory distress syndrome (ARDS) and/or need for mechanical ventilation. All participants are treated with alisporivir+SOC or SOC alone for 14 days.

The study population consists in adults (18-80 years old) hospitalised for 4E3 hours prior to randomisation with a diagnosis of COVID-19 based on symptoms onset and SARS-CoV-2 RT-PCR test positive from nasopharyngeal swab.

In this study, a laboratory-confirmed SARS-CoV-2 infection is determined by reverse transcription polymerase chain reaction (RT-PCR) in a nasopharyngeal swab. All patients have the RT-PCR COVID-19 test based on the same validated method for the duration of the study in order to ensure data consistency and reliability. Sample collection and processing for determining the viral load is done in a standardised and consistent manner in order to decrease variability between samples that might influence the viral load. Droplet digital PCR is used to quantify the average viral load per infected host cell, accounting for the number of cells from each swabbing sample and the viral RNA copies/cell.

The duration of treatment is 14 days (D1-D14). Treatment may be administered on an inpatient (patient hospitalized, at least until D4) or outpatient (patient discharged from hospital) basis. Patients are screened and, if eligible, randomised 2:1 to one of the two treatment arms:
Treatment Arm 1: Alisporivir+SOC (N=60): Sixty participants enrolled and randomised to receive alisporivir 600 mg p.o. BID (twice daily) plus SOC from D1 to D14. Total duration of treatment: 14 days.
Treatment Arm 2: SOC (N=30): Thirty participants enrolled and randomised to receive SOC

During this Phase 2 study, alisporivir (oral solution) is administered either orally or via a nasogastric tube, if necessary after beginning of treatment (condition of patient does not require the use of a nasogastric tube in enrolment), at the dose of 600 mg p.o. BID from D1 to D14 to patients in Arm 1. The total duration of treatment is 14 days.

The investigator should choose the SOC based on locally accepted regimen protocols for patient care and select agents based on the underlying diagnosis and the severity of COVID-19. Additions or changes to SOC are allowed during the patient participation in the study based on patient status and evolution, including antibiotics, excepting e.g. azithromycin and other antibiotics listed as prohibited medications per protocol. Changes in SOC are allowed and are not considered a treatment failure.

The main objective of this study is to evaluate the reduction in SARS-CoV-2 viral load in nasopharyngeal swabs at Day 7 (D7) in patients hospitalised for COVID-19 and treated either with alisporivir and standard of care (SOC) or SOC alone.

The primary endpoint of this study is Viral Load Response Rate (VLRR), defined as the proportion of patients with an intra patient decrease of 1.5 log 10 viral load, at D7 compared to baseline. Thus the primary outcome measure is the change in SARS-CoV-2 viral load in nasopharyngeal swabs [Time Frame: at Day 1 and Day 7].

The secondary objective of this study is to evaluate the clinical and radiological efficacy, safety and tolerability of alisporivir plus SOC compared to SOC alone in patients with COVID-19. For such evaluation, the secondary outcome measures are as follows:

| | |
|---|---|
| Clinical Efficacy: Percentage of patients | D 1 to hospital |
| reporting each severity rating on an 8-point | discharge and then at |
| ordinal scale (as described in Example 2) | D 7, D 11, EOT and at |
| | both FUP visits |
| Change in NEWS from baseline. NEWS has | D 1 to hospital |
| demonstrated an ability to discriminate patients | discharge and then at |
| at risk of poor outcomes. It is based on 7 clinical | D 7, D 11, EOT and at |
| parameters as described in Example 2. | both FUP visits. |
| Changes in thoracic CT scan at D 14 vs. D 1, at | D 1, EOT, D 90 ± 2 |
| D 90 vs. D 1, at D 90 vs. D 14 assessed as | days |
| “progression”, “stable”, or “regression”, and | |
| comparison of percentages between the two | |
| arms. | |
| Percentage of patients admitted to ICU and | D 1 to D 28 ± 2 days |
| Time to admission to ICU | and to D 90 ± 2 days |
| Percentage of patients requiring mechanical | D 1 to D 28 ± 2 days |
| ventilation | and to D 90 ± 2 days |
| Percentages of patients negative for SARS- | EOT, FUP7 and |
| CoV-2 RNA in nasopharyngeal swabs at D 14 | D 28 ± 2 days |
| (EOT) and FUP visits in the two treatment arms. | |
| Time to negative viral load | D 1 to D 28 ± 2 days |
| Time to resolution of symptoms | D 1 to D 28 ± 2 days |
| | and to D 90 ± 2 days |
| Duration of need for supplemental oxygen | D 1 to D 28 ± 2 days |
| | and to D 90 ± 2 days |
| Duration of hospitalisation - measured in days. | D 1 to D 28 ± 2 days |
| | and to D 90 ± 2 days |
| Duration of new non-invasive ventilation or high | D 1 to D 28 ± 2 days |
| flow oxygen use | and to D 90 ± 2 days |
| Duration of new oxygen use | D 1 to D 28 ± 2 days |
| | and to D 90 ± 2 days |
| Duration of new ventilator or ECMO use | D 1 to D 28 ± 2 days |
| | and to D 90 ± 2 days |
| Number of non-invasive ventilation/high flow | D 1 to D 28 ± 2 days |
| oxygen free days | and to D 90 ± 2 days |
| Number of oxygenation free days | D 1 to D 28 ± 2 days |
| | and to D 90 ± 2 days |
| Patient all-cause mortality at D 14 (EOT visit), at | D 1 to EOT, and to |
| D 28 (FUP14), at D 90 (EOS): Date and cause of | D 28 ± 2 days, and to |
| death (if applicable). | D 90 ± 2 days |
| Safety: Cumulative incidence of any AE | D 1 to D 90 ± 2 days |
| Cumulative incidence of Grade 3 and 4 AEs | D 1 to D 90 ± 2 days |
| Cumulative incidence of SAEs | D 1 to D 90 ± 2 days |

Abbreviations: D 1, D 7, D 28 . . . Day 1, Day 7, Day 28 . . . ; EOT End of Treatment; FUP Follow up; ICU Intensive Care Unit; NEWS National Early Warning Score; AE Adverse Event; SAE Serious Adverse Event

There are daily treatment visits from D1-D4, and at D7. The visits on D5, D6 and D8 to D13 take place only if the patient remains hospitalized. Information may still be collected by telephone if the patient is no longer hospitalized at the time of visit. There is an EOT visit upon completion of the therapy (D14), and three FUP visits at approximately 7 days (FUP7) [i.e. D21 of study+/−2 days], 14 days (FUP14) [i.e. D28 of study+/−2 days] and 76 days (End of Study, EOS) after the completion of therapy [i.e. D90 of study+/−2 days]. SARS-CoV-2 viral load assessment occurs at each visit except EOS and pulmonary clinical assessment occurs at each visit. Other clinical and laboratory assessments are defined per protocol.

Inclusion criteria include:
Adult males and females 1E3 years and <30 years of age at the time of screening.
Are hospitalised during the screening period with duration of hospitalisation prior to randomisation 4E3 hours.
Have a diagnosis of COVID-19 based on symptoms onset and positive

SARS-CoV-2 RT-PCR test from nasopharyngeal swab.
Viral load 30 Ct
Have at least one (1) of the following:
   Radiographic pulmonary infiltrates (CT scan), AND/OR
   Clinical assessment (evidence of rales/crackles on exam) AND SpO2≤94% on room air, AND/OR
   Requirement for supplemental oxygen.

Exclusion criteria include:
Patients with ARDS or patients requiring mechanical ventilation at screening or randomisation.
In the opinion of the investigator, the patient is unlikely to survive the following 7 days after randomisation due to a rapidly progressive or terminal illness with a high risk of mortality due to any cause, including acute hepatic failure, respiratory failure or severe septic shock.
Patients who are unconscious or considered by the investigator unable to consent.
Other severe co-morbidity with life expectancy months according to the investigator's assessment.
Critically ill patients who have an APACHE II score
Concomitant severe bacterial infection including blood stream infections, endocarditis, osteomyelitis, retroperitoneal abscess, septic arthritis, or meningitis diagnosed within 7 days prior to randomisation (bacterial pulmonary infection that may complicate COVID-19 is not an exclusion criterion).
Any of the following signs of severe sepsis:
   Shock or profound hypotension defined as systolic blood pressure 90 mm Hg or a decrease of 40 mm Hg from the value obtained during screening that is not responsive to fluid challenge.
   Hypothermia (core temperature 35.6° C.).
   Disseminated intravascular coagulation (DIC) as evidenced by PT, PTT 2×upper limit of normal (ULN), or platelets ≤50% of the lower limit of normal (LLN).
History of positive test for human immunodeficiency virus (HIV) including all patients currently on highly active antiretroviral therapy (HAART) regardless of the CD4+ cell count.
Presence of immunodeficiency or an immunocompromised condition including neutropenia, haematologic malignancy, history of haematopoietic stem cell transplant, history of solid organ transplant, receiving immunosuppressive therapy and long-term use of systemic corticosteroids.
Severe hepatic impairment at screening, as evidenced by alanine aminotransferase (ALT) or aspartate aminotransferase (AST)≥3×ULN or total bilirubin≥2×ULN (except in case of known Gilbert syndrome), or clinical signs of cirrhosis or end-stage hepatic disease (e.g., ascites, hepatic encephalopathy).
Acute hepatitis, cirrhosis (any Child-Pugh class), acute hepatic failure or acute decompensation of chronic hepatic failure.
Alkaline phosphatase ≥3.0×ULN. Patients with values ≥3.0×ULN and ≤5.0×ULN are eligible if this value is documented to be acute and directly related to the infectious process being treated.
Severe renal impairment (creatinine-clearance ≤30 mL/min) or end-stage renal disease (ESRD) requiring haemodialysis or peritoneal dialysis, according to Cockcroft-Gault.
Uncontrolled hypertension (systolic blood pressure ≥150 mmHg or diastolic blood pressure ≥90 mmHg).
Uncontrolled thyroid function (Thyroid Stimulating Hormone [TSH], free T3 or free T4 outside of normal ranges).
History of known or suspected Clostridium difficile infection.
History of epilepsy or known seizure disorder (excluding a history of childhood febrile seizures).
Acute co-morbidity within 7 days before inclusion such as myocardial infarction.
A female who is pregnant or breastfeeding.
Documented hypersensitivity reaction or anaphylaxis to alisporivir, one of the non-active ingredients or any of the SOC medications.
Receipt of any investigational medication in the 3 months prior to screening.
Anticipated transfer to another hospital that is not a study site during the first 4 days of treatment.
Patients previously treated with antivirals, immunomodulators (mAbs in the 3 months prior to screening) and other medicines prohibited in this study in the 14 days prior to randomisation.
Ongoing or recent use of any other medication (including over the counter medication and herbal products) within 14 days before randomisation or within 5 drug half-lives of that medication (whichever is longer) that are known inhibitors/inducers of cytochrome P450 3A or P-glycoprotein (P-gp), or inhibitors of organic anion transporting polypeptides (OATPs), multi resistance protein 2 (MRP2) or bile salt export pump (BSEP).
Known need of concomitant treatment with the following medications during treatment with alisporivir and 14 days after the end of treatment:
   Known inhibitors/inducers of cytochrome P450 3A or P-gp, or inhibitors of OATPs, MRP2 or BSEP;
   Drugs with narrow therapeutic index that are known sensitive substrates of cytochrome P450 3A, or substrates of P-gp, OATPs, MRP2 or BSEP.
Any other condition or prior therapy, which, in the opinion of the investigator, would make the patient unsuitable for this study.
Patients with history of pancreatic disease.

1. A method for treating or preventing a human patient suffering of COVID-19 infections comprising administering Alisporivir to said human patient.

2. The method according to claim 1, wherein said COVID-19 infections are due to SARS-CoV-2.

3. The method according to claim 1 wherein said step of administering Alisporivir reduces SARS-CoV-2 viral load.

4. The method according to claim 2, wherein said SARS-CoV-2 is located in airways of the human.

5. The method according to claim 1 wherein said Alisporivir is administered at a total dose comprised between 200 mg and 1500 mg per day.

6. The method according to claim 1 wherein said Alisporivir is administered at a total dose comprised between 400 mg and 1200 mg per day.

7. The method according to claim 1 wherein said Alisporivir is administered at a total dose comprised between 800 mg and 1200 mg per day.

8. The method according to claim 1 wherein the alisporivir is administered once a day, or twice a day, or three times a day, or four times a day.

9. The method according to claim 1 wherein the Alisporivir is administered at a daily regimen of 800 mg to 1200 mg as total dose per day, one or two times a day.

10. The method according to claim 1 wherein the Alisporivir is administered through oral route or naso-gastric intubation route.

11. The method according to claim 1 wherein the Alisporivir is administered daily for up to 21 days.

12. The method according to claim 1 wherein the Alisporivir is administered daily for up to 14 days.

13. The method of claim 1 wherein said human patient is suffering from medium to severe COVID-19 infections.

14. The method according to claim 13, wherein said human patient meets the following criteria:
a) has a diagnosis of COVID-19 infection with an onset of symptoms and a positive PCR test within 48 hours prior hospitalisation;
and any of the following criteria:
b) radiographic infiltrates by imaging;
c) clinical assessment and SpO2<94% on room air; or
d) requiring supplemental oxygen.
